(19) United States
(12) Patent Application Publication
(10) Pub. No.: US 2012/0276012 A1
(43) Pub. Date: Nov. 1, 2012

(54) Method of Treatment and Screening Method

(75) Inventors: Craig Lygate, Oxford (GB); Stefan Neubauer, Oxford (GB)

(73) Assignee: ISIS INNOVATION LIMITED, Oxford, Oxfordshire (GB)

(21) Appl. No.: 13/508,391

(22) PCT Filed: Nov. 11, 2010
(86) PCT No.: PCT/GB10/51884

§ 371 Date: Jul. 19, 2012

(30) Foreign Application Priority Data
Nov. 13, 2009 (GB) 0919837.5

(51) Int. Cl. A61K 31/711; C12N 15/85; A61P 9/10; C12N 5/077; A61K 49/00; A61P 9/00; C12Q 1/02; G01N 24/00

(52) U.S. Cl. 424/9.2; 435/29; 435/320.1; 435/375; 514/44 R

(57) ABSTRACT

A method for the prevention or treatment of a cardiovascular disease comprising increasing the intracellular creatine concentration in myocardial cells, in particular by up-regulating the creatine transporter. Also described are screening methods for identifying substances to be used in the prevention or treatment of a cardiovascular disease.

### FIELD OF THE INVENTION

The present invention relates to methods for preventing and treating cardiovascular diseases, such as ischemia, reperfusion injury, coronary heart disease, myocardial infarction and angina pectoris, by increasing the intracellular creatine concentration in the heart and, in particular, by modulating the creatine transporter. The invention also relates to a screening method for identifying substances useful for the treatment of these conditions.

### BACKGROUND OF THE INVENTION

ATP is the direct source of energy for all mechanical work in the heart, and is mainly produced by oxidative phosphorylation in the mitochondria. The creatine kinase (CK) energy shuttle is an energy transfer mechanism delivering the energy in ATP from mitochondria to sites of energy utilisation such as the myofibrils. Mitochondrial creatine kinase catalyses the transfer of the high-energy phosphate bond in ATP to creatine (Cr) to form ADP and phosphocreatine (PCr)—which is the main energy storage molecule in the heart and is available for regeneration of ATP during periods of ischemia or high workload. Phosphocreatine rapidly diffuses from the mitochondria to the myofibrils where myofibrillar creatine kinase catalyses the reformation of ATP from phosphocreatine. The released free creatine then diffuses back to the mitochondria. Under normal circumstances approximately one third of the total creatine pool exists as free creatine and two thirds as phosphocreatine. The creatine kinase system acts as an energy buffer by lowering phosphocreatine levels in order to maintain ATP at a normal level when energy demand exceeds energy supply.

Creatine is produced by the liver and kidneys and transported to the heart, where it is taken up by a specific plasma membrane creatine transporter (CrT) against a 50-fold concentration gradient. The uptake and resulting intracellular creatine concentration ([Cr]) are tightly controlled, for example oral creatine supplementation results in increased plasma creatine concentration but has no effect on myocardial creatine levels due to down-regulation of the cardiac creatine uptake capacity. Specifically, down-regulation of the CrT in response to elevated plasma creatine concentration acts to maintain intracellular creatine concentration at normal values. Due to this tight regulation it has proved very difficult to increase the myocardial creatine concentration to supranormal levels, although this has been achieved by genetic overexpression of the creatine transporter. Creatine transporter activity appears to be regulated by gene expression and also, predominantly, by other mechanisms.

It has been suggested to use creatine in various therapies to increase energy provision. For example, oral supplementation with creatine may be effective in attenuating the degenerative state in some muscle disorders (e.g. Duchenne), central nervous system disorders (e.g. Parkinson's) and bone and metabolic disturbances (e.g. osteoporosis). Creatine supplementations are also widely used to improve athletic performance in high-intensity sports such as weight-lifting.

The health care burden of coronary heart disease (CHD) and of chronic heart failure (CHF) in the UK is enormous: CHD is the most common cause of death, with one in six affected by the disease and 2 million people suffering from chronic angina (www.heartstats.org). CHF affects ˜1.8m people, and prognosis is poor (40% mortality in the first year after diagnosis (Dayer & Cowie Clin Med. 2004; 4:13-18)). The economic impact is equally huge, with per annum costs of several billion pounds for CHD and £716m for CHF (www.heartstats.org). Even with optimal medical therapy, mortality and morbidity remain high for both conditions (Graham et al. Eur J Cardiovasc Prey Rehabil. 2007; 14 Suppl 2:S1-113; Dickstein et al. Eur J Heart Fail. 2008; 10(10):933-989), and new, more effective forms of treatment are urgently needed.

In heart failure there are various changes in cardiac energy metabolism. For example, although myocardial ATP levels remain normal until the advanced stages of the disease, both phosphocreatine and total creatine levels decrease at an earlier stage and to a greater extent. Downregulation of the CrT function contributes to these reduced levels. Further, the activities of both mitochondrial and myofibrillar creatine kinases decrease. However, elevating myocardial creatine levels as a treatment is not obvious since mice that were genetically engineered to overexpress the CrT only in the heart and which have very high levels of creatine developed hallmarks of cardiac hypertrophy and failure (Wallis et al. Circulation. 2005; 112:3131-3139).

Current treatment of heart failure is primarily based on inhibition of neurohormonal systems, such as the adrenergic and renin-angiotensin-aldosterone systems. Even with the best treatment, mortality and morbidity remain high, and new therapeutic approaches are required.

New therapeutic approaches are required for other cardiovascular diseases, in particular those that involve periods of ischemia. Current practice during most cardiac surgery procedures involve the use of cardioplegia to arrest the heart, in order to make the operation easier and to lower metabolic demand and preserve ATP. This is often combined with cooling the heart.

Worldwide, there are millions of angina sufferers in whom revascularisation is either not feasible or only partially relieves symptoms. Improved medical therapy for this debilitating condition is urgently needed. Current pharmacological treatments for angina either reduce metabolic demand by decreasing frequency and force of contraction (e.g. Ca²⁺ channel blockers, beta-blockers) or increase blood flow to the heart by vasodilatory mechanisms (e.g. nitrates, K⁺ channel activators).

It is an object of the present invention to provide an alternative strategy for treating such cardiovascular diseases. Specifically it is an object of the invention to elevate energy stores in the heart to protect against, or improve tolerance to, periods of ischaemia and/or conditions of increased energy demand.

It is a further object of the invention to provide screening methods for identifying compounds that alter myocardial creatine levels for use in the treatment of these conditions.

### SUMMARY OF THE INVENTION

According to a first aspect the invention provides a method for the prevention or treatment of a cardiovascular disease comprising increasing the intracellular creatine concentration in myocardial cells. Advantageously, this method provides a new therapeutic strategy which is not currently exploited by any available therapeutic agent. This will also allow combination therapies with existing drugs which have very different mechanisms of action.

In one embodiment, the method comprises increasing intracellular creatine concentration in myocardial cells by up-regulation of the creatine transporter, for example by increasing the expression and/or activity of the creatine transporter. Preferably the intracellular creatine concentration in myocardial cells may be increased by administration of a small molecule modulator of the creatine transporter.

Preferably the intracellular creatine concentration in myocardial cells is increased by 20% to 100% over normal levels.

Preferably the cardiovascular disease is selected from ischemia, reperfusion injury, coronary heart disease, myocardial infarction and angina pectoris. The invention may also be used for protecting against ischemia-reperfusion injury in the heart, for example where the heart is a hypertrophied or failing heart, and/or prior to cardiac surgery or cardiac transplantation.

According to a second aspect the invention provides a vector comprising a creatine transporter gene for the prevention or treatment of a cardiovascular disease. The invention also provides a pharmaceutical composition comprising a vector comprising a creatine transporter gene for the prevention or treatment of a cardiovascular disease, and the use of a vector comprising a creatine transporter gene for the manufacture of a medicament for the prevention or treatment of a cardiovascular disease.

According to a further aspect the invention provides a method for screening for a substance, or a salt or a solvate thereof, to be used in the prevention or treatment of a cardiovascular disease, which comprises the following steps:
(i) providing cells which express a creatine transporter gene;
(ii) incubating the cells with labelled creatine, non-radiolabelled creatine and a test substance;
(iii) measuring the radio-signal attributable to intracellular radiolabel; and
(iv) comparing radiolabel uptake in the presence of the test substance with radiolabel uptake in the absence of the test substance, wherein an increase in uptake indicates that the test substance may be useful in the prevention or treatment of cardiovascular disease.

The invention provides a further method for screening for a substance, or a salt or a solvate thereof, to be used in the prevention or treatment of a cardiovascular disease, which comprises the following steps:
(i) providing cells which express a creatine transporter gene;
(ii) incubating the cells with creatine and a test substance;
(iii) measuring the intracellular creatine levels in vitro using ¹H-MRS; and
(iv) comparing creatine uptake in the presence of the test substance with creatine uptake in the absence of the test substance, wherein an increase in uptake indicates that the test substance may be useful in the prevention or treatment of cardiovascular disease.

The invention also provides a method for screening for a substance, or a salt or a solvate thereof, to be used in the prevention or treatment of a cardiovascular disease, which comprises the following steps:
(i) administering a test substance to a non-human animal; and
(ii) measuring the intracellular creatine concentration in the heart of said non-human animal, by ¹H-MRS, after administration of the test substance.
(iii) comparing the intracellular creatine concentration in the heart of said non-human animal before administration of the test substance with intracellular creatine concentration in the heart of said non-human animal after administration of the test substance, wherein an increase of the intracellular creatine concentration indicates that the substance administered may be useful in the prevention or treatment of the cardiovascular disease.

Advantageously, these methods may provide novel therapeutics for the treatment of cardiovascular diseases, such as ischemia, reperfusion injury, coronary heart disease, myocardial infarction and angina pectoris.

### BRIEF DESCRIPTION OF THE DRAWINGS

FIG. 1 shows myocardial injury following in vivo ischemia/reperfusion is lower in mouse hearts within the therapeutic [Cr] range of 83-140 nmol/mg protein.

FIG. 2 shows negative correlation between myocardial creatine concentration and myocardial injury following in vivo ischemia/reperfusion.

FIG. 3 shows heart function (recovery of rate pressure product, RPP) in isolated perfused mouse hearts before, during, and after 20 mins of global ischemia (from time=10 mins to 30 mins). Diamond symbols represent control wildtype mice and square symbols transgenic mice overexpressing the CrT.

FIG. 4 shows PCr concentration in isolated perfused mouse hearts before, during, and after 20 mins of global ischemia. Square symbols represent control wildtype mice and diamond symbols transgenic mice overexpressing the CrT.

FIG. 5A shows how the uptake of ¹⁴C-labelled Cr by a culture of fibroblast 3T3 cells that stably express the CrT varies with extracellular Cr concentration

FIG. 5B shows dose-dependent inhibition of creatine uptake by β-GPA in the same 3T3 cell line, and in HL1 cells incubated with 250 μM creatine. This validates that the screening method is sensitive enough to detect small changes in creatine uptake in response to pharmacological modulation.

### DETAILED DESCRIPTION OF THE INVENTION

The invention relates to a novel method for the prevention or treatment of cardiovascular diseases, such as ischemia, reperfusion injury, coronary heart disease, myocardial infarction and angina pectoris, by increasing the intracellular creatine concentration in the heart and, in particular, by modulating the creatine transporter. It is shown herein that mice over-expressing the creatine transporter (CrT-OE mice) have elevated [Cr] and [PCr] in the heart, increasing the energy buffering capacity, and protecting against acute ischemia/reperfusion injury. This proof-of-principle data suggests the therapeutic potential of pharmacological agents capable of modulating intracellular [Cr], e.g. by modulating activity of the creatine transporter (CrT) or its endogenous regulators.

The invention provides a new approach to treating cardiovascular diseases, by providing the heart with higher energy stores before an ischemic insult and thereby reducing ischemic damage and/or angina etc. Specifically the levels of creatine in the heart are increased, such that when an ischemic insult occurs, the heart has larger energy reserves to fall back on before the detrimental effects of ischemia can take effect. This is in contrast to current therapies which increase blood supply to the heart or lower oxygen and/or nutrient demand; in other words they aim to correct the ischemic damage after it has occurred.

The conditions that may be prevented or treated in accordance with the invention include cardiovascular diseases, such as ischemia, reperfusion injury, coronary heart disease, myocardial infarction, angina pectoris and heart failure. In general, the terms ‘prevent’ and ‘treat’ encompass the prevention of the development of a disease or a symptom in a patient who may have a predisposition of the disease or the symptom but has not yet been diagnosed to have the disease or the symptom; the inhibition of the symptoms of a disease, namely, inhibition or retardation of the progression thereof; and the alleviation of the symptoms of a disease, namely, regression of the disease or the symptoms, or inversion of the progression of the symptoms.

As used herein, the term “cardiovascular diseases” refers to the class of diseases that involve the heart or blood vessels (arteries and veins). Cardiovascular diseases in which patients experience episodes of ischemia and/or reperfusion injury are of particular interest in relation to the invention, as are cardiovascular diseases in which the subject's heart experiences conditions of increased energy demand. Ischemia is an absolute or relative shortage of the blood supply to an organ, which may result in a shortage of oxygen, glucose and other blood-borne fuels. A relative shortage means the mismatch of blood supply (oxygen/fuel delivery) and demand for adequate metabolism of tissue. Ischemia results in tissue damage because of a lack of oxygen and nutrients, which if prolonged leads to cell death. Ischemia may be caused by constriction or blockage of the blood vessels supplying a part of the body resulting in an inadequate flow of blood to it. The heart, the kidneys, and the brain are among the organs that are the most sensitive to inadequate blood supply. Ischemia in brain tissue may be caused by stroke or head injury and may ultimately kill brain tissue. By ‘prevent’ or ‘treat’ ischemia it is meant that the duration of the ischemic episode is reduced and/or the ischemic episode is reversed or prevented altogether, thereby reducing damage to or protecting the organ in question.

The invention is particularly concerned with ischemic heart disease (IHD), or myocardial ischemia, which is a disease characterized by reduced blood supply to the heart muscle, usually due to coronary artery disease (or coronary heart disease, which refers to failure of coronary circulation to supply adequate circulation to cardiac muscle and surrounding tissue, for example due to atherosclerosis of the coronary arteries). Ischemic heart disease may present with various symptoms, including angina pectoris (chest pain on exertion, in cold weather or emotional situations); acute chest pain: acute coronary syndrome, angina pectoris or myocardial infarction (heart attack), and heart failure (difficulty in breathing or swelling of the extremities due to weakness of the heart muscle).

By ‘prevent’ or ‘treat’ ischemic heart disease it is meant that the duration of the ischemic episode is reduced and/or the ischemic episode is reversed or prevented altogether, thereby reducing damage to or protecting the heart. By ‘prevent’ myocardial infarction it is meant the chronic treatment as secondary protection for patients at high risk of myocardial infarction. By ‘prevent’ or ‘treat’ angina pectoris it is meant that the symptoms of angina are reduced and/or the angina threshold may be shifted to higher workload, i.e. patients may be able to be more active before angina occurs.

Restoration of blood flow after a period of ischemia can actually be more damaging than the ischemia itself. Reperfusion injury refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function. In the heart, myocardial reperfusion injury may be caused by the rapid flow of blood into areas previously rendered ischemic by coronary artery occlusion resulting in arrhythmia, infarction and/or myocardial stunning Ischemia-reperfusion injury is a complex phenomenon often encountered in surgical practice. By ‘prevent’ ischemia-reperfusion injury it is meant that the degree of damage to tissue upon reperfusion is reduced or prevented. By ‘treat’ ischemia-reperfusion injury it is meant that any damage caused to tissue by reperfusion is reversed, such that normal function of the tissue is restored, and/or a more rapid recovery of normal tissue heart function is observed than in the absence of treatment.

Specific uses of the invention include treatment of the following indications:

i) Protection against ischemia/reperfusion injury in hearts, including hypertrophied and failing hearts. For example, the invention may be used as an acute treatment prior to cardiac surgery (such as bypass grafts or valve replacement), i.e. to load hearts with Cr before cardioplegia, providing more energy to spend during whole heart ischemia, thereby protecting the heart from damage.
ii) Chronic treatment as secondary protection for patients at high risk of myocardial ischemia and infarction.
iii) Chronic treatment of angina—with more energy to expend, the angina threshold may be shifted to higher workload, i.e. patients may be able to be more active before angina occurs.
iv) Treatment of heart failure. In this aspect, combination therapy is likely to be particularly effective.
v) Dosing ‘healthy’ organ donors a few days before harvest—in patients that have been declared dead on the basis of absent brain-stem activity, and where consent has been obtained for transplantation of their organs, treatment to elevate myocardial creatine levels prior to explanation of the heart.

The inventive method may also be beneficial in analogous brain conditions such as ischemic stroke or transient ischemic attacks. Other potential applications include treatment of muscular dystrophies to build skeletal muscle strength.

### Creatine and the Creatine Transporter

The present invention provides for increasing the intracellular creatine concentration in myocardial cells in order to prevent or treat the various conditions mentioned herein. The intracellular creatine concentration may be increased by any means, for example the use of creatine esters as described in US 2007/0203076.

Preferably the intracellular creatine concentration is increased by up-regulation of the creatine transporter. By up-regulation of the creatine transporter it is meant that the expression and/or activity of the creatine transporter is increased. The expression of the CrT may be increased by gene therapy with a construct which provides for the expression of a CrT. For example, Wallis et al. describe a transgenic mouse in which the myocardial CrT is overexpressed (Circulation 2005; 112:3131-3139). Snow and Murphy (Molecular and Cellular Biochemistry (2001) 224:169-191) provide a review of creatine and the creatine transporter.

Any CrT gene may be expressed, including human (Gene ID 6535; NC_{—}000023.10), mouse (GeneID 102857; NC_{—}000086.6) or rat genes (Gene ID 50690; NC_{—}005120.2), the sequences of which may be found at the National Center for Biotechnology Information, NIH database (http://www.ncbi.nlm.nih.gov/sites/entrez), or conservatively modified variants thereof.

The term ‘conservatively modified variants’ is one well known in the art and indicates variants containing changes which are substantially without effect on protein function. For example, it includes polypeptide or amino acid sequences which are derived from a particular starting polypeptide or amino acid sequence and which share a sequence identity that is about 80%, 85%, 86%, 87%, 88%, 89%, 90%, 91%, 92%, 93%, 94%, 95%, 96%, 97%, 98%, 99%, with the particular starting polypeptide or amino acid sequence. The term is also conveniently defined as found in U.S. Pat. No. 5,380,712 which is incorporated herein by reference for such purpose. It is well recognised in the art that the replacement of one amino acid in a peptide or protein with another amino acid having similar properties, for example the replacement of a glutamic acid residue with an aspartic acid residue, may not substantially alter the properties or structure of the peptide or protein in which the substitution or substitutions were made.

A gene transfer vector available for gene therapy is well known in the present technical field, and it can be selected, as appropriate, depending on a gene introduction method or a host. Examples of such a vector include an adenovirus vector, adeno-associated virus, lenti-virus and a retrovirus vector. When a CrT gene is ligated to a gene transfer vector, a control sequence such as a promoter or a terminator, a signal sequence, a polypeptide-stabilizing sequence, etc. may be appropriately ligated, such that the gene can be expressed in a host. Selection or construction of such vectors is well known to the skilled person. Expression of the CrT may be constitutive or inducible. For example, the CrT gene may be under the control of a response element, such as a tetracycline response element, whereby administration of tetracycline controls CrT expression (and thereby creatine dose). Alternatively, the promoter may be a cardiac-specific promoter, such as the alpha-MHC promoter.

Alternatively or additionally, the activity of the CrT may be increased, either by genetic modification to a more active form or by the administration of one or more substances to an individual which cause increased activity of the CrT, for example a small molecule modulator of the creatine transporter. Such a small molecule modulator of the CrT is preferably a molecule which is able to pass through the transporter into the cell, like creatine, but is able to inhibit substrate feedback. In other words, such molecules have a high affinity to block the Cr regulatory site on the CrT or associated regulatory protein, preventing negative feedback that would normally occur as a result of rising intracellular Cr concentrations. Such molecules could be termed “CrT activators” in that they increase the activity of the transporters, thereby allowing more creatine to enter the cell and allowing increased concentrations of creatine to be achieved in the cell.

### Administration

In the treatment comprising the administration of a small molecule modulator of the creatine transporter, such substance may be mixed with a pharmaceutically acceptable carrier, so that it can be provided in the form of a pharmaceutical composition. Any ratio of active ingredient to carrier may be used and the ratio of an active ingredient to a carrier may preferably be between 1% and 90% by weight. In addition, the pharmaceutical composition of the present invention can be administered to humans or organisms other than humans [for example, non-human mammals (e.g. a bovine, a monkey, a chicken, a cat, a mouse, a rat, a hamster, a swine, a canine, etc.), birds, reptiles, amphibians, fish, insects, etc.] in various forms via either an oral administration route (including gavage, or admixture with food or drink) or a parenteral administration route (e.g. intravenous injection, intramuscular injection, subcutaneous administration, rectal administration, intraperitoneal, and dermal administration). Accordingly, as the pharmaceutical composition of the present invention, an active ingredient can be used singly. However, it is also possible to formulate such an active ingredient with a pharmaceutically acceptable carrier or diluent by a method commonly used depending on an administration route, so as to manufacture a formulation having a suitable dosage form. Any dosage form known to the skilled person may be used. Any appropriate carrier or diluent may be used, for example isotonic saline solution, buffers, etc. Such pharmaceutical carriers are well known in the art and the selection of a suitable carrier is deemed to be within the scope of those skilled in the art from the teachings contained herein.

The small molecule modulator of the creatine transporter is administered in vivo in an amount effective to effect an increase in the activity of the CrT and produce an increase in the intracellular concentration of creatine, most preferably an increase of 20 to 100% above normal levels. Myocardial creatine levels may be measured by any method known to the skilled person, but preferably they may be quantified non-invasively by ¹H-MRS, as described in Schneider et al. (Magnetic Resonance in Medicine, 2004, 52:1029-1035). ¹H-MRS may be carried out in animals for in vivo drug screening purposes and in man to monitor therapeutic levels (e.g. for treatment optimisation). The term ‘an effective amount’ for purposes of this application shall mean that amount of substance capable of producing the desired effect. The amount of substance which is given depends upon a variety of factors including the age, weight and condition of the patient, the administration route, the properties of the pharmaceutical composition, the condition of the patient, the judgment of a doctor, the condition and the extent of treatment or prevention desired. The substance may be administered to the individual as a short-term therapy or long-term therapy depending on the condition and the extent of treatment or prevention desired. For example, short-term treatment (e.g. by transient gene therapy) may have prolonged effects due to slow in vivo degradation of myocardial creatine.

In the mouse model described below, a therapeutic range of 83-140 nmol/mg protein has been found to be effective, i.e. a beneficial effect was observed on I/R injury with 20% increase in the intracellular concentration of creatine above normal levels (i.e. 83 nmol/mg protein). These measurements may be made in homogenised tissue by HPLC, with the creatine concentration normalised to the total protein content of the sample (which is measured by a standard spectrophotometric assay). Above 100% increase in the intracellular concentration of creatine above normal levels (which is 140 nmol in mouse) toxic effects were observed, i.e. the hypertrophy and heart failure described in Wallis (2005). It will be apparent to the skilled person how to determine a therapeutic range in humans or other animals. By aiming for an elevation in the intracellular concentration of creatine of 20-100% above normal levels, one can obtain a therapeutic range which will form the basis for determining dosing of any putative CrT activating compound. For example, with the gene therapy aspect of the invention the level of creatine may be maintained within the therapeutic range by controlling expression of the CrT gene, for example by putting it under the control of an inducible promoter.

The small molecule modulator of the creatine transporter may be employed alone or in combination with other techniques, drugs or compounds for preventing or treating cardiovascular diseases, such as ischemia, reperfusion injury, coronary heart disease, myocardial infarction, angina pectoris and heart failure. Elevation of [Cr] represents a new therapeutic strategy which is not currently exploited by any available therapeutic agent. This has the advantage of having benefits that are additive to existing drugs which have very different mechanisms of action. As examples:—

(i) Current practice for cardioplegia involves cooling the heart to lower metabolic demand and preserve ATP. Our novel strategy would be additive to this by increasing the energy reserve available for regeneration of used ATP.
(ii) Current pharmacological treatments for angina either reduce metabolic demand by decreasing frequency and force of contraction (e.g. Ca²⁺ channel blockers, beta-blockers) or increase blood flow to the heart by vasodilatory mechanisms (e.g. nitrates, K+ channel activators). Elevation of [Cr] represents a novel approach by making the heart less sensitive to the brief periods of ischemia that occur during angina attacks.
(iii) Current treatment of heart failure is primarily based on inhibition of neurohormonal systems, such as the adrenergic and renin-angiotensin-aldosterone systems. Even with the best treatment, mortality and morbidity remain high, and new therapeutic approaches are required. Elevation of [Cr] represents a unique approach to maintain normal cardiac energetic in the failing heart.

Accordingly, small molecule modulator of the creatine transporter may be administered in combination with one or more of the above-mentioned treatments.

In the gene therapy of the present invention, it may be possible to select either an in vivo method of directly administering a recombinant vector encoding the gene of interest to a patient, or an ex vivo method of collecting a target cell from a patient body, introducing a CrT gene, or a recombinant vector encoding the gene of interest, or DNA constructs carrying agonist encoding genes e.g. heavy and light chains of an engineered agonist antibody, into the target cell outside of the body, and returning the target cell, into which the aforementioned gene or vector has been introduced, to the patient body. In the case of the in vivo method, the recombinant vector encoding the gene of interest is directly administered to a patient by using a gene transfer vector known in the present technical field, such as a retrovirus vector. As with the pharmaceutical composition of the present invention, such a CrT gene used in the gene therapy of the present invention, or a gene transfer vector to which the CrT gene is operably linked, can be mixed with a pharmaceutically acceptable carrier, so as to produce a formulation. Such a formulation can be parenterally administered, for example. The formulation could alternatively be delivered by IV injection, for example by direct myocardial injection (e.g. during coronary artery bypass surgery for secondary prevention), or released directly into coronary arteries during catheterization. Fluctuation of a dosage level can be adjusted by standard empirical optimizing procedures, which are well understood in the present technical field. An alternative for in vivo administration is to use physical approaches, such as particle bombardment or jet injection, to directly deliver DNA encoding heavy and light chains of an engineered agonist antibody (Walther et al Mol Biotechnology 28:121-128, 2004; Yang et at PNAS 87:9568-72, 1990). In the case of the ex vivo method, such a CrT gene can be introduced into a target cell according to a method known in the present technical field, such as the calcium phosphate method, the electroporation method, or the viral transduction method. In the case of selecting the ex vivo method, a CrT gene or a gene transfer vector to which the CrT gene is operably linked is introduced into a cell, preferably a myocardial cell, and the aforementioned peptide is then allowed to express in the cell. Thereafter, the cell is transplanted to a patient, so that a cardiovascular disease can be treated.

Up-regulation of the creatine transporter by gene therapy preferably produces an increase in the intracellular concentration of creatine, most preferably an increase of 20 to 100% above normal levels, as measured, for example, by ¹H-MRS. It may be possible to control the up-regulation of the CrT (e.g. increase of activity and/or expression) by using an inducible promoter or control element. For example, by placing the CrT gene under the control of a response element, such as a tetracycline response element, CrT expression (and thereby creatine dose) may be controlled by administration of tetracycline.

### Screening Method

The invention also provides methods for identifying small molecular modulators of the CrT which may be used as pharmacological tools or potential therapeutic agents in the treatment of the cardiovascular diseases described herein. The targeting strategy is to modulate the CrT as this is the sole mechanism by which creatine can enter the cell, involving active uptake against a large concentration gradient. Feeding extra dietary creatine can elevate [Cr] in skeletal muscle, but does not alter [Cr] in cardiomyocytes due to consequent down-regulation of the CrT. Indeed, negative feedback by creatine itself is the only verified regulator of the cardiac CrT, and the screening strategy therefore exploits this.

In one aspect the invention provides a method for screening for a substance, or a salt or a solvate thereof, to be used in the prevention or treatment of a cardiovascular disease, which comprises the following steps:
(i) providing cells which express a creatine transporter gene;
(ii) incubating the cells with labelled creatine, non-labelled creatine and a test substance;
(iii) measuring the signal attributable to intracellular label; and
(iv) comparing label uptake in the presence of the test substance with label uptake in the absence of the test substance, wherein an increase in uptake indicates that the test substance may be useful in the prevention or treatment of cardiovascular disease.

The invention provides a further method for screening for a substance, or a salt or a solvate thereof, to be used in the prevention or treatment of a cardiovascular disease, which comprises the following steps:
(i) providing cells which express a creatine transporter gene;
(ii) incubating the cells with creatine and a test substance;
(iii) measuring the intracellular creatine levels in vitro using ¹H-MRS; and
(iv) comparing creatine uptake in the presence of the test substance with creatine uptake in the absence of the test substance, wherein an increase in uptake indicates that the test substance may be useful in the prevention or treatment of cardiovascular disease. This alternative measurement method involves exposing cells to non-labelled creatine, with incubation and exposure to test substance as before. Cells are then washed and lysed before measurement of creatine levels in vitro using ¹H-MRS.

Any molecules may be subjected to these screening methods although preferably, molecules with similar chemical features to creatine are selected for testing. Molecules may be selected using a variety of computational approaches (for example substructure searching, similarity searching and shape-based screening). Using creatine itself as a query molecule, a library may be mined for compounds sharing common or similar structural features, as assessed by Tanimoto coefficients—a commonly employed shape comparison parameter.

In a more detailed analysis, multiple conformers may be generated from creatine and these conformers may be overlaid (using ROCS) with conformers generated from each of the library members. Hits may be ranked according to their Tanimoto coefficients and “Scaled Colour” (a measure of alignment of similar functional groups), and subsequently assessed in their ranking order. These methods should enable the efficient identification of candidate pharmacological probes. The proposed existence of different binding sites on the CrT for creatine as substrate and creatine as regulator will enable selectivity to be engineered through modifications to the structure of the creatine analogues to provide a creatine analogue that can enter the cell via the CrT, but has higher affinity to block Cr regulatory site, preventing negative feedback that would normally occur as a result of rising intracellular Cr concentrations. This is directly analogous to the closely related glutamine transporter (SLC38 System A), in which acute regulation of activity occurs via direct substrate inhibition, i.e. reduced amino acid substrate increases transporter activity. A secondary phase via transcriptional regulation occurs over longer periods. It is likely that regulation of the CrT is similar, with both acute and chronic mechanisms.

The assays described herein are sensitive and specific assays for measuring radiolabelled Cr uptake in cell culture using cells that express a CrT gene. The cells which express a CrT gene may be prepared in any manner known to the skilled person, for example as described in Mortensen R M and Kingston R E (Unit 9.5, Current protocols in Molecular Biology 2009; John Wiley and Sons Inc., DOI: 10.1002/0471142727.mb0905s86), or Wang Z et al. (Canc Res 2008; 68:492-7), or Hocevar B A et al. (J Biol Chem 2005; 280(27):25920-7). Preferably the CrT gene is expressed under the control of a constitutive promoter, for example the cardiac-specific alpha-myosin heavy chain (a-MHC) promoter or the cytomegalovirus (CMV) minimal promoter. Alternatively the cells may endogenously express a CrT, which may be determined, for example, by RT-PCR. Any CrT gene may be expressed, including human (Gene ID 6535; NC_{—}000023.10), mouse (GeneID 102857; NC_{—}000086.6) or rat genes (Gene ID 50690; NC_{—}005120.2) the sequences of which may be found at the National Center for Biotechnology Information, NIH database using a search tool (http://www.nebi.nlm.nih.gov/,sites/entrez), or conservatively modified variants thereof.

Any cell type may be used in accordance with the invention, for example NIH3T3 murine fibroblasts, Human Embryonic Kidney HEK293 and Chinese Hamster Ovary CHO cells, Monkey COS-7 (simian CV-1 in Origin, and carrying the SV40 genetic material). Preferred are cardiomyocyte derived cell lines which endogenously express the CrT or cells with a cardiomyocyte-like phenotype such as HL-1 cells, or H9c2 cells derived from rat ventricular heart tissue (Shin et al., 2009 J Cell Physiol, 221:490-497). Alternatively, a primary culture of isolated cardiomyocytes may be used. Preferably the isolated cardiomyocytes are from mammals. Cells are plated and incubated to form monolayers in any manner known to the skilled person. For example 1×10⁵ cells may be plated into 24-well plates and incubated for 18 hours to form monolayers.

According to the first screening method, the cells are incubated with labelled creatine, non-labelled creatine and a test substance. Preferably the creatine is radiolabelled creatine, for example ¹⁴C-labelled creatine or ³H-labelled creatine (R S Carling et al. Ann Clin Biochem 2008; 45:575-584). Alternatively the creatine may be labelled with a fluorescent label (a fluorophore). Preferably the cells are incubated with about 10 μl (37 kBq) radiolabelled Cr (0-500 μM), about 500 μM (25-1000 μM) non-radiolabelled Cr and about 30 μM (0-100 μM) test compound.

After a suitable period of incubation as may be readily determined by the skilled person (e.g. 37° C. for 60 min (95% air, 5% CO₂)), media may be aspirated, cells solubilised and lysed (e.g. using PBS/TritonX-100). A scintillation counter measures signal attributed to intracellular radiolabel, with Cr uptake estimated against known standards. Control wells will contain no test compound to measure normal background uptake, and control wells containing only cells will be used for protein quantification. Experiments are preferably performed in triplicate.

When comparing radiolabel uptake in the presence of the test substance with radiolabel uptake in the absence of the test substance, an increase in uptake indicates that the test substance may be useful in the prevention or treatment of cardiovascular disease. Preferably compounds will be considered positive in this in vitro screen if uptake is >2 standard deviations higher than the mean for control wells. These compounds may be re-tested in vitro to determine dose dependency, and to confirm involvement of the CrT by including Cr-uptake inhibitors (β-GPA or chloride-free buffer). This will differentiate between compounds that alter creatine uptake via alternative mechanisms, e.g. changing membrane potential, pH, or altered Na⁺ handling.

The screening method may be optimised by the skilled person using his common general knowledge. The screen has been validated by demonstrating dose-dependent inhibition of creatine uptake by the creatine analogue β-guanidinopropionic acid (β-GPA as set out in Example 3 below, to confirm sensitivity to small changes in creatine uptake.

According to the second screening method, the cells are incubated with creatine (non-labelled) and a test substance. Preferably the cells are incubated with about 500 μM (25-1000 μM) non-labelled Cr and about 30 μM (0-100 μM) test compound. The cells are then washed and lysed as described above, before measurement of creatine levels in vitro using ¹H-MRS.

A further assay may comprise measuring ¹⁴C-creatine uptake in the presence of test compound in Langendorff perfused heart experiments (as in Ten Hove et al. 2008 J. Molecular and Cellular Cardiology 45:453-459).

Ultimately, compounds may be tested in vivo, with hearts excised after 1-5 days of compound administration for creatine determination by HPLC. In vivo ¹H-MRS may be used to measure myocardial [Cr] non-invasively (as in Schneider et al. 2004 Magnetic Resonance in Medicine 52: 1029-1035), allowing the performance of dose-response studies in the same animal longitudinally.

In a further aspect, the invention provides a method for screening for a substance, or a salt or a solvate thereof, to be used in the prevention or treatment of a cardiovascular disease, which comprises the following steps:
(i) administering a test substance to a non-human animal;
(ii) measuring the intracellular creatine concentration in the heart of said non-human animal, by ¹H-MRS, after administration of the test substance; and
(iii) comparing the intracellular creatine concentration in the heart of said non-human animal before administration of the test substance with intracellular creatine concentration in the heart of said non-human animal after administration of the test substance, wherein an increase of the intracellular creatine concentration indicates that the substance administered may be useful in the prevention or treatment of the cardiovascular disease.

Preferably the animal is a rodent, such as a mouse or a rat. The animal may be a healthy animal or may have a cardiovascular disease. For example, standard surgical models known to the skilled person may be used to cause ischemia/reperfusion injury, or chronic heart failure. Alternatively, pharmacological agents may be administered to cause heart failure (e.g. doxorubicin or sympathomimetic drugs), or to cause coronary vasospasm (e.g. vasopressin).

In the screening method of the present invention, the intracellular creatine concentration before administration (e.g. by feeding) of a test substance is compared with the intracellular creatine concentration after administration of the test substance. When the intracellular creatine concentration is increased as compared to the former levels, it can be determined that the test substance is useful for the treatment of the disease. The creatine concentration may be measured in vivo by ¹H-MRS or ex vivo by HPLC.

Alternatively or additionally, if the animal is showing symptoms of a cardiovascular disease then these can also be measured and compared before and after administration of the test substance. The symptom may be measured:
by echocardiography or MRI, e.g. ejection fraction, fractional shortening, stroke volume, cardiac output, ventricular volumes and LV mass;
by LV catheterisation e.g. left ventricular systolic and diastolic pressures, first derivatives of these with respect to time (i.e. dP/dtmax and dP/dtmin), and time-to-peak parameters; also recovery of these or of LV developed pressure following I/R injury;
by ECG, ST segment changes e.g. time to onset and duration as measures of ischemia;
by plasma markers of CV disease e.g. CK, LDH, BNP.

The type of a substance screened by the screening method of the present invention is not particularly limited. Any molecules may be subjected to screening although preferably, molecules with similar chemical features to creatine are selected for testing (CrT activators), as discussed above.

The test substance may be administered to the non-human animal by any route and dosing regime, which may be established by standard experimentation, as will be apparent to the skilled person. Preferably the dosing regime comprises chronic treatment (days to weeks) to pre-elevate myocardial [Cr], and may include dosing with oral creatine supplement to enhance rate of uptake. Preferably the substance is administered orally.

### EXAMPLES

The following examples are illustrative of the products and methods falling within the scope of the present invention. They are not to be considered in any way limitative of the invention.

### Mice Over-Expressing the Creatine Transporter (CrT-OE Mice) have Elevated [Cr] and [PCr] in the Heart

### Example 1

In this example it is shown that mice over-expressing the creatine transporter (CrT-OE mice) have elevated [Cr] and [PCr] in the heart, increasing the energy buffering capacity, and protecting against acute ischemia/reperfusion injury.

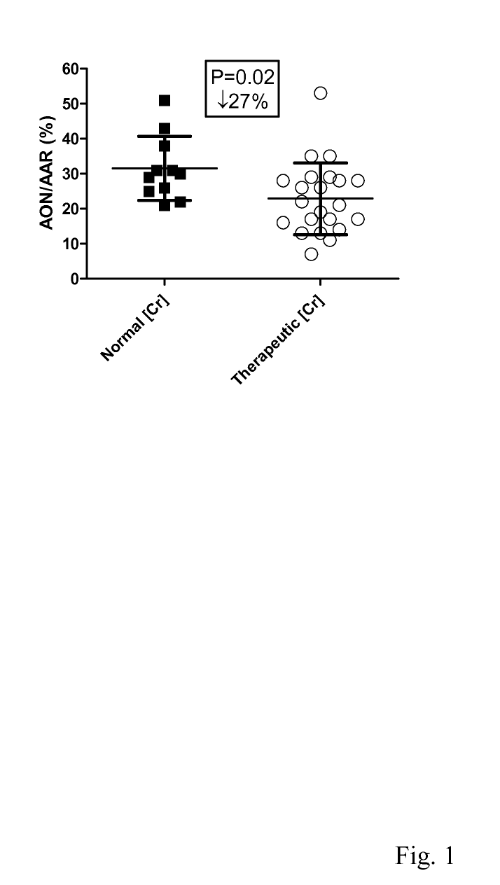

In vivo experiments have been performed to surgically create regional Ischemia in the mouse heart. The coronary artery of CrT-OE mice was occluded for 45 minutes in vivo, followed by reperfusion for 24 hours. Hearts were then excised and stained histologically to quantify the extent of myocardial damage. This is expressed as the ratio of area-of-necrosis (AON) to area-at-risk (AAR), i.e. the fraction of the Ischemic area that has died (Bohl et al. Am J Physiol 2009:D0100836.02009, online publication). Myocardial [Cr] was measured by HPLC in the right ventricle at the end of the experiment (Ten Hove et al. 2008 J. Molecular and Cellular Cardiology 45:453-459). A therapeutic range was predefined as myocardial [Cr] of 83-140 nmol/mg protein, equating to an increase [Cr] of ˜20-100% over control mice (levels above this are associated with toxicity (Wallis et al. Circulation. 2005; 112:3131-3139)). These mice demonstrated 27% less myocardial injury than control mice with normal [Cr]. FIG. 1 shows myocardial injury following in vivo ischemia/reperfusion is lower in mouse hearts within the therapeutic [Cr] range of 83-140 nmol/mg protein.

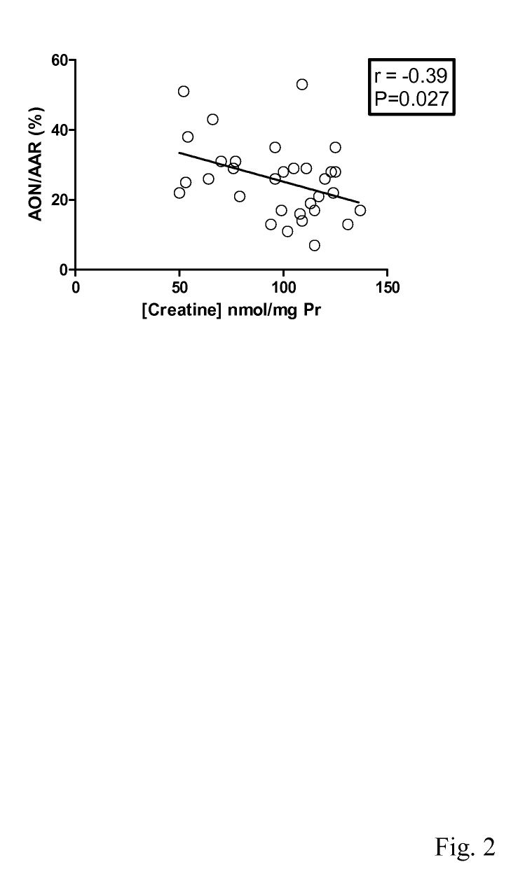

Furthermore, reduced myocardial injury correlated with high [Cr]. FIG. 2 shows negative correlation between myocardial creatine concentration and myocardial injury following in vivo ischemia/reperfusion. This suggests a dose-dependent protective effect of creatine against ischemia/reperfusion injury.

### Example 2

In a separate experiment, hearts from CrT-OE mice were perfused ex vivo and systolic function measured as rate pressure product (the product of developed pressure and heart rate). Simultaneous measurements of [PCr] were obtained using magnetic resonance spectroscopy (as in ten Hove et al. 2005 Circulation 111: 2477-2485). CrT-OE mice had elevated [PCr] but identical baseline function compared to wildtype controls. When global ischemia was simulated by stopping the perfusate, function ceased in all mice.

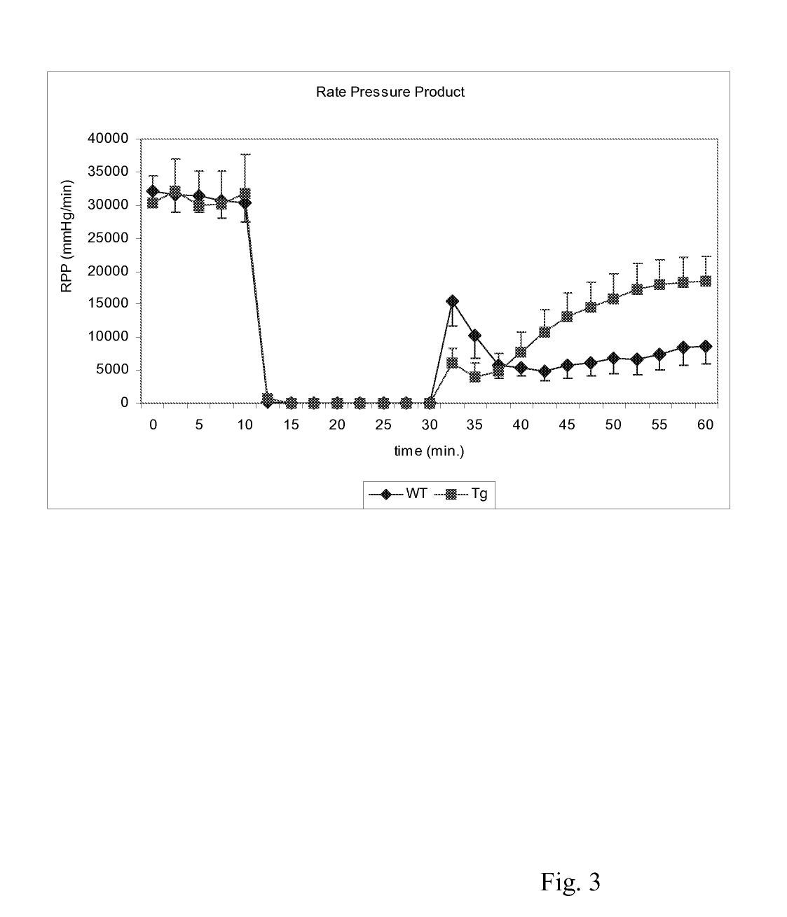

Following global ischemia, recovery of heart function on reperfusion was significantly better in the CrT-OE mice. FIG. 3 shows heart function (RPP) in isolated perfused mouse hearts before, during, and after 20 mins of global ischemia (from time=10 mins to 30 mins). Diamond symbols represent control wildtype mice and square symbols transgenic mice overexpressing the CrT. Previous work has shown that very high myocardial [Cr] is detrimental, however, levels <140 nmol are well tolerated in follow-up studies of CrT-OE mice up to 18 months of age. This suggests that moderately elevated myocardial creatine is safe even over long periods of time.

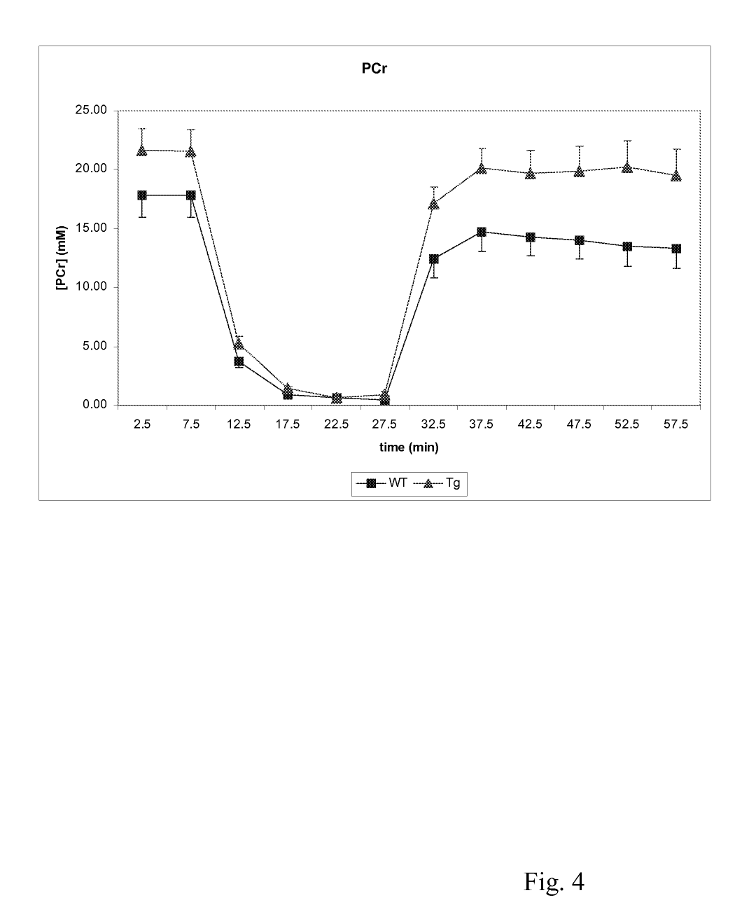

Myocardial concentration of PCr was confirmed to be higher at baseline in Tg mice and was regenerated more rapidly after reperfusion. FIG. 4 shows PCr concentration in isolated perfused mouse hearts before, during, and after 20 minutes of global ischemia. Square symbols represent control wildtype mice and diamond symbols transgenic mice overexpressing the CrT.

The data suggests that the beneficial effect observed in I/R injury is likely to be a combination of several factors:

i) In the cardiomyocyte, creatine is phosphorylated via the action of creatine kinase to form phosphocreatine (PCr), which is the main energy storage molecule in the heart. The heart can only store enough ATP to last for a few beats after blood supply is interrupted, and at such times PCr acts as an energy buffer to regenerate ATP. Elevating [Cr] results in more PCr, increasing the energy buffering capacity so that it will take longer from the onset of ischemia for ATP to be depleted.
ii) Elevated [Cr] prior to ischemia may result in faster regeneration of PCr.
iii) Elevated creatine inhibits mitochondrial permeability transition pore (MPTP) formation, reducing apoptosis and cellular damage.
iv) Creatine has an effect to protect against oxidative stress.

### Example 3

### Identification of Compounds that Increase Creatine Uptake Via CrT

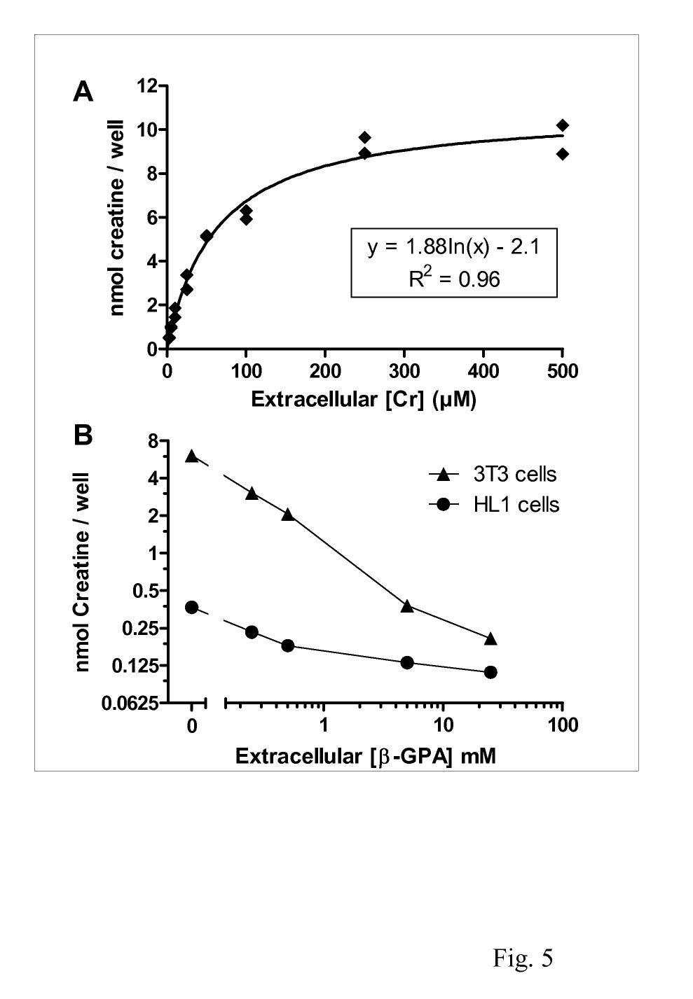

We have developed a sensitive and specific assay for measuring ¹⁴C-labelled Cr uptake in cell culture using fibroblast 3T3 cells that stably express the CrT. Cells (1×10⁵ cells) were plated into 24-well-plates, and incubated for 18 hours to form monolayers. Each well was spiked with 10 μl (37 kBq) ¹⁴C-creatine, 500 μM of non-radiolabelled Cr, and 30 μM of test compound (β-guanidinopropionic acid) (performed in triplicate). After incubation at 37° C. for 60 min (95% air, 5% CO₂), media was aspirated, cells solubilised and lysed using PBS/TritonX-100. A scintillation counter measures signal attributed to intracellular radiolabel, with Cr uptake estimated against known standards. Control wells contain no test compound to measure normal background uptake, and control wells containing only cells are used for protein quantification. FIG. 5A shows how the uptake of ¹⁴C-labelled Cr by a culture of fibroblast 3T3 cells that stably express the CrT varies with extracellular Cr concentration.

HL-1 cells that are derived from mouse atrial tumour cells and have a beating cardiomyocyte-like phenotype. It was confirmed that these cells express endogenous CrT using RT-PCR. Changes in creatine uptake were measured using the ¹⁴C screen described above.

FIG. 5B demonstrates dose-dependent inhibition of creatine uptake by the creatine analogue β-guanidinopropionic acid (β-GPA), in both 3T3 cells and HL1 cells. In both cases as the extracellular concentration of β-GPA was increased, the creatine uptake by the cells was observed to decrease. This result was expected as β-GPA is a competitive antagonist for creatine uptake by the CrT. The results confirm the sensitivity of the assay to small changes in creatine uptake.

### Example 4

### Elevating Myocardial Creatine is Safe in Mice with Chronic Heart Failure Following Myocardial Infarction

In this experiment CrT-OE mice were pre-selected based on myocardial creatine levels measured in vivo by ¹H-MRS (as in Schneider et al. Magnetic Resonance in Medicine, 2004, 52:1029-1035). Mice with creatine 20-100% above normal baseline values, and a group of control mice with normal myocardial creatine levels, underwent surgical occlusion of the left anterior descending coronary artery to induce chronic myocardial infarction. After 6 weeks there was no difference in survival between groups, and left ventricular (LV) remodelling and function were compared by cine-MRI and LV haemodynamics as previously described (ten Hove et al. 2005 Circulation 111: 2477-2485). All mice had changes in cardiac structure and function indicative of chronic heart failure, however, when matched for infarct size, no significant differences were observed for any parameter of LV function between normal and elevated myocardial creatine groups (e.g. for ejection fraction, cardiac output, LV end-diastolic pressure or contractile reserve). These results suggest that creatine elevation (at least as mono-therapy) is not effective in chronic heart failure. However, they also demonstrate that transgenic over-expression of the CrT was sufficient to maintain creatine levels at supra-physiological levels throughout the protocol despite the development of chronic heart failure and that moderate Cr elevations are safe in the failing heart. This is important, since patients with pre-existing heart failure are prime candidates for cardiac surgery, and therefore for cardio-protection by elevating creatine.

### Example 5

### Chronic Elevation of Myocardial Creatine to Moderate Levels is Safe

We have previously shown that very high levels of myocardial creatine cause LV hypertrophy and dysfunction in CrT-OE mice (Wallis et al. Circulation. 2005; 112:3131-3139). However it is unknown whether chronic exposure to moderately elevated creatine levels has a similar effect. To determine this we performed cardiac ultrasound and LV haemodynamic measurements in CrT-OE and wildtype controls at 80 weeks of age. Myocardial creatine was measured in heart tissue post-mortem using HPLC to create two experimental groups—mice with normal myocardial creatine (average 76 nmol Cr/mg protein) and mice with moderate elevation of creatine (maximum of 140 nmol Cr/mg protein). No significant differences were observed between groups for any parameter e.g. LV/body weight, LV pressures, contractility (dP/dtmax) or relaxation (tau). This demonstrates that long-term elevation of myocardial creatine to moderate levels is not detrimental to the heart.

1. A method for the prevention or treatment of a cardiovascular disease comprising increasing the intracellular creatine concentration in myocardial cells.

2. A method for protecting against ischemia-reperfusion injury in the heart of a subject comprising increasing the intracellular creatine concentration in myocardial cells of the subject.

3. A method according to claim 1 wherein the intracellular creatine concentration is increased by up-regulation of the creatine transporter.

4. A method according to claim 3 which comprises increasing the expression and/or activity of the creatine transporter.

5. A method according to claim 3 which comprises administration of a small molecule modulator of the creatine transporter.

6. A method according to claim 5 wherein the small molecule modulator of the creatine transporter is able to block the Cr regulatory site on the CrT or associated regulatory protein, thereby preventing negative feedback that would normally occur as a result of rising intracellular Cr concentrations.

7. A method according to claim 1 wherein the cardiovascular disease is selected from ischemia, reperfusion injury, coronary heart disease, myocardial infarction and angina pectoris.

8. A method according to claim 2 wherein the heart is a hypertrophied or failing heart.

9. A method according to claim 2 which is an acute treatment prior to cardiac surgery or cardiac transplantation.

10. A method according to claim 1 wherein the intracellular creatine concentration in myocardial cells is increased by 20% to 100% over normal levels.

11. A vector comprising a creatine transporter gene for the prevention or treatment of a cardiovascular disease.

12. A pharmaceutical composition comprising a vector comprising a creatine transporter gene for the prevention or treatment of a cardiovascular disease.

13. A method for the prevention or treatment of a cardiovascular disease comprising administering a therapeutically effective amount of a vector comprising a creatine transporter gene.

14. A method for screening for a substance, or a salt or a solvate thereof, to be used in the prevention or treatment of a cardiovascular disease, which comprises the following steps:
(i) providing cells which express a creatine transporter gene;
(ii) incubating the cells with labelled creatine, non-labelled creatine and a test substance;
(iii) measuring the signal attributable to intracellular label; and
(iv) comparing label uptake in the presence of the test substance with label uptake in the absence of the test substance, wherein an increase in uptake indicates that the test substance may be useful in the prevention or treatment of cardiovascular disease.

15. A method for screening for a substance, or a salt or a solvate thereof, to be used in the prevention or treatment of a cardiovascular disease, which comprises the following steps:
(i) providing cells which express a creatine transporter gene;
(ii) incubating the cells with creatine and a test substance;
(iii) measuring the intracellular creatine levels in vitro using ¹H-MRS; and
(iv) comparing creatine uptake in the presence of the test substance with creatine uptake in the absence of the test substance, wherein an increase in uptake indicates that the test substance may be useful in the prevention or treatment of cardiovascular disease.

16. A method according to claim 14 wherein the cells are fibroblast 3T3 cells over-expressing the CrT or HL-1 cells.

17. A method for screening for a substance, or a salt or a solvate thereof, to be used in the prevention or treatment of a cardiovascular disease, which comprises the following steps:
(i) administering a test substance to a non-human animal; and
(ii) measuring the intracellular creatine concentration in the heart of said non-human animal, by ¹H-MRS, after administration of the test substance.
(iii) comparing the intracellular creatine concentration in the heart of said non-human animal before administration of the test substance with intracellular creatine concentration in the heart of said non-human animal after administration of the test substance, wherein an increase of the intracellular creatine concentration indicates that the substance administered may be useful in the prevention or treatment of the cardiovascular disease.

18. A method according to claim 2 wherein the intracellular creatine concentration is increased by up-regulation of the creatine transporter.

19. A method according to claim 18 which comprises increasing the expression and/or activity of the creatine transporter.

20. A method according to claim 4 which comprises administration of a small molecule modulator of the creatine transporter.

21. A method according to claim 15 wherein the cells are fibroblast 3T3 cells over-expressing the CrT or HL-1 cells.
